SEQUENCE LISTING (22 sequences)

SEQ ID NO: 1 - protein, 344 aa, Homo sapiens
MKRASAGGSRLLAWVLWLQAWQVAAPCPGACVCYNEPKVTTSCPQQGLQAVPVGIPAASQRIFLHGNRISHVPAASFRACRNLTILWLHSNVLARIDAAAFTGLALLEQLDLSDNAQLRSVDPATFHGLGRLHTLHLDRCGLQELGPGLFRGLAALQYLYLQDNALQALPDDTFRDLGNLTHLFLHGNRISSVPERAFRGLHSLDRLLLHQNRVAHVHPHAFRDLGRLMTLYLFANNLSALPTEALAPLRALQYLRLNDNPWVCDCRARPLWAWLQKFRGSSSEVPCSLPQRLAGRDLKRLAANDLQGCAVATGPYHPIWTGRATDEEPLGLPKCCQPDAADKA

SEQ ID NO: 2 - protein, 344 aa, Rattus
MKRASSGGSRLPTWVLWLQAWRVATPCPGACVCYNEPKVTTSRPQQGLQAVPAGIPASSQRIFLHGNRISYVPAASFQSCRNLTILWLHSNALAGIDAAAFTGLTLLEQLDLSDNAQLRVVDPTTFRGLGHLHTLHLDRCGLQELGPGLFRGLAALQYLYLQDNNLQALPDNTFRDLGNLTHLFLHGNRIPSVPEHAFRGLHSLDRLLLHQNHVARVHPHAFRDLGRLMTLYLFANNLSMLPAEVLVPLRSLQYLRLNDNPWVCDCRARPLWAWLQKFRGSSSGVPSNLPQRLAGRDLKRLATSDLEGCAVASGPFRPFQTNQLTDEELLGLPKCCQPDAADKA

SEQ ID NO: 3 - protein, 285 aa, Homo sapiens
PCPGACVCYNEPKVTTSCPQQGLQAVPVGIPAASQRIFLHGNRISHVPAASFRACRNLTILWLHSNVLARIDAAAFTGLALLEQLDLSDNAQLRSVDPATFHGLGRLHTLHLDRCGLQELGPGLFRGLAALQYLYLQDNALQALPDDTFRDLGNLTHLFLHGNRISSVPERAFRGLHSLDRLLLHQNRVAHVHPHAFRDLGRLMTLYLFANNLSALPTEALAPLRALQYLRLNDNPWVCDCRARPLWAWLQKFRGSSSEVPCSLPQRLAGRDLKRLAANDLQGCA

SEQ ID NO: 4 - protein, 319 aa, Homo sapiens
PCPGACVCYNEPKVTTSCPQQGLQAVPVGIPAASQRIFLHGNRISHVPAASFRACRNLTILWLHSNVLARIDAAAFTGLALLEQLDLSDNAQLRSVDPATFHGLGRLHTLHLDRCGLQELGPGLFRGLAALQYLYLQDNALQALPDDTFRDLGNLTHLFLHGNRISSVPERAFRGLHSLDRLLLHQNRVAHVHPHAFRDLGRLMTLYLFANNLSALPTEALAPLRALQYLRLNDNPWVCDCRARPLWAWLQKFRGSSSEVPCSLPQRLAGRDLKRLAANDLQGCAVATGPYHPIWTGRATDEEPLGLPKCCQPDAADKA

SEQ ID NO: 5 - protein, 284 aa, Rattus
CPGACVCYNEPKVTTSRPQQGLQAVPAGIPASSQRIFLHGNRISYVPAASFQSCRNLTILWLHSNALAGIDAAAFTGLTLLEQLDLSDNAQLRVVDPTTFRGLGHLHTLHLDRCGLQELGPGLFRGLAALQYLYLQDNNLQALPDNTFRDLGNLTHLFLHGNRIPSVPEHAFRGLHSLDRLLLHQNHVARVHPHAFRDLGRLMTLYLFANNLSMLPAEVLVPLRSLQYLRLNDNPWVCDCRARPLWAWLQKFRGSSSGVPSNLPQRLAGRDLKRLATSDLEGCA

SEQ ID NO: 6 - protein, 318 aa, Rattus
CPGACVCYNEPKVTTSRPQQGLQAVPAGIPASSQRIFLHGNRISYVPAASFQSCRNLTILWLHSNALAGIDAAAFTGLTLLEQLDLSDNAQLRVVDPTTFRGLGHLHTLHLDRCGLQELGPGLFRGLAALQYLYLQDNNLQALPDNTFRDLGNLTHLFLHGNRIPSVPEHAFRGLHSLDRLLLHQNHVARVHPHAFRDLGRLMTLYLFANNLSMLPAEVLVPLRSLQYLRLNDNPWVCDCRARPLWAWLQKFRGSSSGVPSNLPQRLAGRDLKRLATSDLEGCAVASGPFRPFQTNQLTDEELLGLPKCCQPDAADKA

SEQ ID NO: 7 - protein, 22 aa, Artificial Sequence
  Synthetic antibody
AAAFTGLTLLEQLDLSDNAQLR

SEQ ID NO: 8 - protein, 10 aa, Artificial Sequence
  Synthetic antibody
LDLSDNAQLR

SEQ ID NO: 9 - protein, 10 aa, Artificial Sequence
  Synthetic antibody
LDLSDDAELR

SEQ ID NO: 10 - protein, 11 aa, Artificial Sequence
  Synthetic antibody
LDLASDNAQLR

SEQ ID NO: 11 - protein, 11 aa, Artificial Sequence
  Synthetic antibody
LDLASDDAELR

SEQ ID NO: 12 - protein, 11 aa, Artificial Sequence
  Synthetic antibody
LDALSDNAQLR

SEQ ID NO: 13 - protein, 11 aa, Artificial Sequence
  Synthetic antibody
LDALSDDAELR

SEQ ID NO: 14 - protein, 11 aa, Artificial Sequence
  Synthetic antibody
LDLSSDNAQLR

SEQ ID NO: 15 - protein, 11 aa, Artificial Sequence
  Synthetic antibody
LDLSSDEAELR

SEQ ID NO: 16 - protein, 12 aa, Artificial Sequence
  Synthetic antibody
DNAQLRVVDPTT

SEQ ID NO: 17 - protein, 6 aa, Artificial Sequence
  Synthetic antibody
DNAQLR

SEQ ID NO: 18 - protein, 16 aa, Artificial Sequence
  Synthetic antibody
ADLSDNAQLRVVDPTT

SEQ ID NO: 19 - protein, 16 aa, Artificial Sequence
  Synthetic antibody
LALSDNAQLRVVDPTT

SEQ ID NO: 20 - protein, 16 aa, Artificial Sequence
  Synthetic antibody
LDLSDNAALRVVDPTT

SEQ ID NO: 21 - protein, 16 aa, Artificial Sequence
  Synthetic antibody
LDLSDNAQLHVVDPTT

SEQ ID NO: 22 - protein, 16 aa, Artificial Sequence
  Synthetic antibody
LDLSDNAQLAVVDPTT

(19) United States
(12) Patent Application Publication
(10) Pub. No.: US 2008/0045926 A1
(43) Pub. Date: Feb. 21, 2008

(54) Treatment of Conditions Involving Dopaminergic Neuronal Degeneration Using Nogo Receptor Antagonists

(76) Inventors: Jane K. Relton, Belmont, MA (US); Thomas M. Engber, Acton, MA (US)

Correspondence Address: STERNE, KESSLER, GOLDSTEIN & FOX, P.L.L.C., 1100 NEW YORK AVE., N.W., WASHINGTON, DC, 20005, US

(21) Appl. No.: 10/587,714

(22) PCT Filed: Jan. 28, 2005
(86) PCT No.: PCT/US05/02535

§ 371 Date: Jun. 4, 2007

Related U.S. Application Data
(60) Provisional application No. 60/540,798, filed on Jan. 30, 2004.

(51) Int. Cl. A61K 38/17

(52) U.S. Cl. 604/522

(57) ABSTRACT

The invention provides methods for promoting regeneration or survival of dopaminergic neurons in a mammal displaying signs or symptoms of dopaminergic neuronal degeneration, including a human with Parkinson's disease, using Nogo receptor antagonists.

### FIELD OF THE INVENTION

This invention relates to neurobiology and pharmacology. More particularly, it relates to methods of treating conditions involving dopaminergic neuronal degeneration by the administration of Nogo receptor-1 antagonists.

### BACKGROUND OF THE INVENTION

Certain neurodegenerative disorders are characterized by degeneration of dopaminergic neurons. For example, Parkinson's disease is associated with progressive destruction of dopaminergic neurons in the substantia nigra of the midbrain. This destruction results in reduced levels of the chemical transmitter dopamine. Physical symptoms of Parkinson's disease include impairment of voluntary movement and uncontrollable rhythmic twitching of groups of muscles producing characteristic shaking.

The most widely used treatment for Parkinson's disease is administration of a dopamine precursor, L-dopa (L-3,4-dihydroxyphenylalanine), which acts indirectly by replacing the missing dopamine. However, disadvantages are associated with the use of L-dopa. Patients often suffer from side effects such as dyskinesia, nausea, vomiting, abdominal distension and psychiatric side effects and patients typically become less responsive to L-dopa treatment over time. Alternative forms of therapy using postsynaptic dopamine agonists also are associated with side effects. Further, although L-dopa treatment improves quality of life for patients, it does not halt disease progression.

Other compounds, such as glial-cell-line-derived neurotrophic factor (GDNF), have shown promise in the treatment of Parkinson's disease in human patients when delivered by chronic infusion. See, e.g., Gill et al., “Direct brain infusion of glial cell line-derived neurotrophic factor in Parkinson disease,” Nature Med. 9: 589-95 (2003). However, these treatment regimens are still in the early stages of development.

Many other diseases and disorders may involve degeneration of dopaminergic neurons. These include multiple system atrophy, striatonigral degeneration, olivopontocerebellar atrophy, Shy-Drager syndrome, motor neuron disease with parkinsonian features, Lewy body dementia, progressive supranuclear palsy, cortical-basal ganglionic degeneration, frontotemporal dementia, Alzheimer's disease with parkinsonism, Wilson disease, Hallervorden-Spatz disease, Chediak-Hagashi disease, SCA-3 spinocerebellar ataxia, X-linked dystonia-parkinsonism (DYT3), Huntington's disease (Westphal variant), prion disease, vascular parkinsonism, cerebral palsy, repeated head trauma, postencephalitic parkinsonism and neurosyphilis.

Accordingly, there remains a need for additional treatment methods for Parkinson's disease and other conditions characterized by degeneration of dopaminergic neurons.

### SUMMARY OF THE INVENTION

The invention relates to a method of treatment of conditions involving dopaminergic neuronal degeneration, including Parkinson's disease, by the administration of Nogo receptor-1 antagonists.

In some embodiments, the invention provides a method of promoting regeneration or survival of dopaminergic neurons in a mammal displaying signs or symptoms of dopaminergic neuronal degeneration, comprising administering to the mammal a therapeutically effective amount of an NgR1 antagonist.

In some embodiments, the NgR1 antagonist is administered directly into the central nervous system. In some embodiments, the NgR1 antagonist is administered directly into the substantia nigra or the striatum. In some embodiments, the NgR1 antagonist is administered by bolus injection or chronic infusion.

In some embodiments, the NgR1 antagonist comprises a soluble form of a mammalian NgR1. In some embodiments, the soluble form of a mammalian NgR1 comprises amino acids 26 to 310 of human NgR1 (SEQ ID NO: 3) with up to ten conservative amino acid substitutions and lacks both a functional transmembrane domain and a functional signal peptide. In some embodiments, the soluble form of a mammalian NgR1 comprises amino acids 26 to 344 of human NgR1 (SEQ ID NO: 4) with up to ten conservative amino acid substitutions and lacks both a functional transmembrane domain and a functional signal peptide. In some embodiments, the soluble form of a mammalian NgR1 comprises amino acids 27 to 310 of rat NgR1 (SEQ ID NO: 5) with up to ten conservative amino acid substitutions and lacks both a functional transmembrane domain and a functional signal peptide. In some embodiments, the soluble form of a mammalian NgR1 comprises amino acids 27 to 344 of rat NgR1 (SEQ ID NO: 6) with up to ten conservative amino acid substitutions and lacks both a functional transmembrane domain and a functional signal peptide.

In some embodiments, the soluble form of a mammalian NgR1 further comprises a fusion moiety. In some embodiments, the fusion moiety is an immunoglobulin moiety. In some embodiments, the immunoglobulin moiety is an Fc moiety.

In some embodiments, the NgR1 antagonist used in the methods of the invention comprises an antibody or antigen-binding fragment thereof that binds to a mammalian NgR1. In some embodiments, the antibody is selected from the group consisting of a polyclonal antibody, a monoclonal antibody, a Fab fragment, a Fab′ fragment, a F(ab′)2 fragment, an Fv fragment, an Fd fragment, a diabody, and a single-chain antibody.

In some embodiments, the antibody or antigen-binding fragment thereof binds to an polypeptide bound by a monoclonal antibody produced by a hybridoma selected from the group consisting of: HB 7E11 (ATCC® accession No. PTA-4587), HB 1H2 (ATCC® accession No. PTA-4584), HB 3G5 (ATCC® accession No. PTA-4586), HB 5B10 (ATCC® accession No. PTA-4588) and HB 2F7 (ATCC® accession No. PTA-4585). In some embodiments, the monoclonal antibody is produced by the HB 7E11 hybridoma. In some embodiments, the antibody or antigen-binding fragment thereof binds to a polypeptide comprises an amino acid sequence selected from the group consisting of:

| | |
|---|---|
| AAAFGLTLLEQLDLSDNAQLR; | (SEQ ID NO: 7) |
| LDLSDNAQLR; | (SEQ ID NO: 8) |
| LDLSDDAELR; | (SEQ ID NO: 9) |
| LDLASDNAQLR; | (SEQ ID NO: 10) |
| LDLASDDAELR; | (SEQ ID NO: 11) |
| LDALSDNAQLR; | (SEQ ID NO: 12) |
| LDALSDDAELR; | (SEQ ID NO: 13) |
| LDLSSDNAQLR; | (SEQ ID NO: 14) |
| LDLSSDEAELR; | (SEQ ID NO: 15) |
| DNAQLRVVDPTT; | (SEQ ID NO: 16) |
| DNAQLR; | (SEQ ID NO: 17) |
| ADLSDNAQLRVVDPTT; | (SEQ ID NO: 18) |
| LALSDNAQLRVVDPTT; | (SEQ ID NO: 19) |
| LDLSDNAALRVVDPTT; | (SEQ ID NO: 20) |
| LDLSDNAQLHVVDPTT; | (SEQ ID NO: 21) |
| and | |
| LDLSDNAQLAVVDPTT. | (SEQ ID NO: 22) |

In some embodiments, the therapeutically effective amount of a NgR1 antagonist used in the methods of the invention is from 0.001 mg/kg to 10 mg/kg. In some embodiments, the therapeutically effective amount is from 0.01 mg/kg to 1.0 mg/kg. In some embodiments, the therapeutically effective amount is from 0.05 mg/kg to 0.5 mg/kg.

In some embodiments, the dopaminergic neuronal degeneration is associated with a disease or disorder selected from the group consisting of Parkinson's disease, multiple system atrophy, striatonigral degeneration, olivopontocerebellar atrophy, Shy-Drager syndrome, motor neuron disease with parkinsonian features, Lewy body dementia, progressive supranuclear palsy, cortical-basal ganglionic degeneration, frontotemporal dementia, Alzheimer's disease with parkinsonism, Wilson disease, Hallervorden-Spatz disease, Chediak-Hagashi disease, SCA-3 spinocerebellar ataxia, X-linked dystonia-parkinsonism (DYT3), Huntington's disease (Westphal variant), prion disease, vascular parkinsonism, cerebral palsy, repeated head trauma, postencephalitic parkinsonism and neurosyphilis.

In some embodiments, the invention provides a method of treating Parkinson's disease, comprising administering to the mammal a therapeutically effective amount of an NgR1 antagonist.

### BRIEF DESCRIPTION OF THE DRAWINGS

FIG. 1A shows dopaminergic neuronal survival in Nogo receptor knockout mice compared to heterozygote and wild-type littermate controls 4 weeks after unilateral injection with 6-hydroxydopamine HCl (6OHDA) injection into the striatum. The number of tyrosine (TH) positive neurons in the injured substantia nigra is expressed as a percentage of the number of TH positive neurons in the contralateral intact substantia nigra. FIG. 1B shows dopaminergic neuronal survival in rats treated with sNgR(310)Fc for 4 weeks after unilateral injection of 6OHDA into the striatum. The number of tyrosine (TH) positive neurons in the injured substantia nigra is expressed as a percentage of the number of TH positive neurons in the contralateral intact substantia nigra.

FIG. 2A shows reduced apomorphine-induced rotational behavior in Nogo receptor knockout mice compared to heterozygote and wild-type littermate controls 4 weeks after unilateral 6OHDA injection into the striatum. FIG. 2B shows reduced amphetamine-induced rotations 7, 14, 21 and 28 days after unilateral 6OHDA injection into the striatum in rats treated with sNgR(310)Fc compared to vehicle-treated controls.

FIG. 3 shows increased striatal dopamine levels in rats treated with sNgR(310)Fc four weeks after unilateral 6OHDA injection into the striatum.

### DETAILED DESCRIPTION OF THE INVENTION

Unless defined otherwise, all technical and scientific terms used herein have the same meaning as commonly understood by one of ordinary skill in the art to which this invention belongs. In case of conflict, the present application including the definitions will control. Also, unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present invention, suitable methods and materials are described below. The materials, methods and examples are illustrative only and are not intended to be limiting. Other features and advantages of the invention will be apparent from the detailed description and from the claims.

Throughout this specification and claims, the word “comprise,” or variations such as “comprises” or “comprising,” indicate the inclusion of any recited integer or group of integers but not the exclusion of any other integer or group of integers.

In order to further define this invention, the following terms and definitions are provided.

As used herein, “antibody” means an intact immunoglobulin, or an antigen-binding fragment thereof. Antibodies of this invention can be of any isotype or class (e.g., M, D, G, E and A) or any subclass (e.g., G1-4, A1-2) and can have either a kappa (κ) or lambda (λ) light chain.

As used herein, “humanized antibody” means an antibody in which at least a portion of the non-human sequences are replaced with human sequences. Examples of how to make humanized antibodies may be found in U.S. Pat. Nos. 6,054,297, 5,886,152 and 5,877,293.

As used herein, a “therapeutically effective amount” refers to an amount effective, at dosages and for periods of time necessary, to achieve the desired therapeutic result.

As used herein, a “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

As used herein, a “patient” means a mammal, e.g., a human.

As used herein, “fusion protein” means a protein comprising a polypeptide fused to another, generally heterologous, polypeptide.

As used herein, a “Nogo receptor antagonist” means a molecule that inhibits the binding of Nogo receptor-1 to a ligand (e.g., NogoA, NogoB, NogoC, MAG, OM-gp).

As used herein, “Nogo receptor polypeptide” includes both full-length Nogo receptor-1 protein and fragments thereof.

The present invention is based on the discovery that treatment with a Nogo receptor antagonist provides improved recovery in dopaminergic pathways after injury and significant improvement in symptoms resulting from dopaminergic neuronal degeneration.

### Nogo Receptor Antagonists

Any Nogo receptor antagonist may be used in the methods of the invention. For example, Nogo receptor antagonists that may be used in the methods of the invention include, but are not limited to: soluble Nogo receptor polypeptides; antibodies to the Nogo receptor protein and antigen-binding fragments thereof; and small molecule antagonists.

### Soluble Nogo Receptor-1 Polypeptides

In some embodiments of the invention, the antagonist is a soluble Nogo receptor-1 polypeptide (Nogo receptor-1 is also variously referred to as “Nogo receptor,” “NogoR,” “NogoR-1,” “NgR,” and “NgR-1”). Full-length Nogo receptor-1 consists of a signal sequence, a N-terminus region (NT), eight leucine rich repeats (LRR), a LRRCT region (a leucine rich repeat domain C-terminal of the eight leucine rich repeats), a C-terminus region (CT) and a GPI anchor. The sequences of full-length human and rat Nogo receptors are shown in Table 1.

TABLE 1

Sequences of Human and Rat Nogo receptor-1
Polypeptides

| | |
|---|---|
| Full-length | MKRASAGGSRLLAWVLWLQAWQVAAPCPGACVCYNEP |
| human | KVTTSCPQQGLQAVPVGIPAASQRIFLHGNRISHVPA |
| Nogo receptor | ASFRACRNLTILWLHSNVLARIDAAAFTGLALLEQLD |
| SEQ ID NO: 1 | LSDNAQLRSVDPATFHGLGRLHTLHLDRCGLQELGPG |
| | LFRGLAALQYLYLQDNALQALPDDTFRDLGNLTHLFL |
| | HGNRISSVPERAFRGLHSLDRLLLHQNRVAHVHPHAF |
| | RDLGRLMTLYLFANNLSALPTEALAPLRALQYLRLND |
| | NPWVCDCRARPLWAWLQKFRGSSSEVPCSLPQRLAGR |
| | DLKRLAANDLQGCAVATGPYHPIWTGRATDEEPLGLP |
| | KCCQPDAADKA |
| Full-length | MKRASSGGSRLPTWVLWLQAWRVATPCPGACVCYNEP |
| rat | KVTTSRPQQGLQAVPAGIPASSQRIFLHGNRISYVPA |
| Nogo receptor | ASFQSCRNLTILWLHSNALAGIDAAAFTGLTLLEQLD |
| SEQ ID NO: 2 | LSDNAQLRVVDPTTFRGLGHLHTLHLDRCGLQELGPG |
| | LFRGLAALQYLYLQDNNLQALPDNTFRDLGNLTHLFL |
| | HGNRIPSVPEHAFRGLHSLDRLLLHQNHVARVHPHAF |
| | RDLGRLMTLYLFANNLSMLPAEVLVPLRSLQYLRLND |
| | NPWVCDCRARPLWAWLQKFRGSSSGVPSNLPQRLAGR |
| | DLKRLATSDLEGCAVASGPFRPFQTNQLTDEELLGLP |
| | KCCQPDAADKA |

Soluble Nogo receptor polypeptides used in the methods of the invention comprise an NT domain; 8 LRRs and an LRRCT domain and lack a signal sequence and a functional GPI anchor (i.e., no GPI anchor or a GPI anchor that fails to efficiently associate to a cell membrane). Suitable polypeptides include, for example, amino acids 26-310 (SEQ ID NO: 3) and 26-344 (SEQ ID NO: 4) of the human Nogo receptor and amino acids 27-310 (SEQ ID NO: 5) and 27-344 (SEQ ID NO: 6) of the rat Nogo receptor (Table 2). Additional polypeptides which may be used in the methods of the invention are described, for example, in International Patent Applications PCT/US02/32007 and PCT/US03/25004.

TABLE 2

Soluble Nogo receptor Polypeptides from Human
and Rat

| | |
|---|---|
| Human 26-310 | PCPGACVCYNEPKVTTSCPQQGLQAVPVGIPAASQRI |
| SEQ ID NO: 3 | FLHGNRISHVPAASFRACRNLTILWLHSNVLARIDAA |
| | AFTGLALLEQLDLSDNAQLRSVDPATFHGLGRLHTLH |
| | LDRCGLQELGPGLFRGLAALQYLYLQDNALQALPDDT |
| | FRDLGNLTHLFLHGNRISSVPERAFRGLHSLDRLLLH |
| | QNRVAHVHPHAFRDLGRLMTLYLFANNLSALPTEALA |
| | PLRALQYLRLNDNPWVCDCRARPLWAWLQKFRGSSSE |
| | VPCSLPQRLAGRDLKRLAANDLQGCA |
| Human 26-344 | PCPGACVCYNEPKVTTSCPQQGLQAVPVGIPAASQRI |
| SEQ ID NO: 4 | FLHGNRISHVPAASFRACRNLTILWLHSNVLARIDAA |
| | AFTGLALLEQLDLSDNAQLRSVDPATFHGLGRLHTLH |
| | LDRCGLQELGPGLFRGLAALQYLYLQDNALQALPDDT |
| | FRDLGNLTHLFLHGNRISSVPERAFRGLHSLDRLLLH |
| | QNRVAHVHPHAFRDLGRLMTLYLFANNLSALPTEALA |
| | PLRALQYLRLNDNPWVCDCRARPLWAWLQKFRGSSSE |
| | VPCSLPQRLAGRDLKRLAANDLQGCAVATGPYHPIWT |
| | GRATDEEPLGLPKCCQPDAADKA |
| Rat 27-310 | CPGACVCYNEPKVTTSRPQQGLQAVPAGIPASSQRIF |
| SEQ ID NO: 5 | LHGNRISYVPAASFQSCRNLTILWLHSNALAGIDAAA |
| | FTGLTLLEQLDLSDNAQLRVVDPTTFRGLGHLHTLHL |
| | DRCGLQELGPGLFRGLAALQYLYLQDNNLQALPDNTF |
| | RDLGNLTHLFLHGNRIPSVPEHAFRGLHSLDRLLLHQ |
| | NHVARVHPHAFRDLGRLMTLYLFANNLSMLPAEVLVP |
| | LRSLQYLRLNDNPWVCDCRARPLWAWLQKFRGSSSGV |
| | PSNLPQRLAGRDLKRLATSDLEGCA |
| Rat 27-344 | CPGACVCYNEPKVTTSRPQQGLQAVPAGIPASSQRIF |
| SEQ ID NO: 6 | LHGNRISYVPAASFQSCRNLTILWLHSNALAGIDAAA |
| | FTGLTLLEQLDLSDNAQLRVVDPTTFRGLGHLHTLHL |
| | DRCGLQELGPGLFRGLAALQYLYLQDNNLQALPDNTF |
| | RDLGNLTHLFLHGNRIPSVPEHAFRGLHSLDRLLLHQ |
| | NHVARVHPHAFRDLGRLMTLYLFANNLSMLPAEVLVP |
| | LRSLQYLRLNDNPWVCDCRARPLWAWLQKFRGSSSGV |
| | PSNLPQRLAGRDLKRLATSDLEGCAVASGPFRPFQTN |
| | QLTDEELLGLPKCCQPDAADKA |

A soluble Nogo receptor polypeptide that is a component of a fusion protein also may be used in the methods of the invention. In some embodiments, the heterologous moiety of the fusion protein is an immunoglobulin constant domain. In some embodiments, the immunoglobulin constant domain is a heavy chain constant domain. In some embodiments, the heterologous polypeptide is an Fc fragment. In some embodiments, the Fc is joined to the C-terminal end of a soluble Nogo receptor polypeptide. In some embodiments, the fusion Nogo receptor protein is a dimer.

### Antibodies

The methods of the invention may be performed using an antibody or an antigen-binding fragment thereof that specifically binds an immunogenic Nogo receptor-1 polypeptide and inhibits the binding of Nogo receptor-1 to a ligand (e.g., NogoA, NogoB, NogoC, MAG, OM-gp). The antibody or antigen-binding fragment used in the methods of the invention may be produced in vivo or in vitro. In some embodiments, the anti-Nogo receptor-1 antibody or antigen-binding fragment thereof is murine or human. In some embodiments, the anti-Nogo receptor-1 antibody or antigen-binding fragment thereof is recombinant, engineered, humanized and/or chimeric. In some embodiments, the antibody is selected from the antibodies described in International Patent Application No. PCT/US03/25004. Antibodies useful in the present invention may be employed with or without modification.

Exemplary antigen-binding fragments of the antibodies which may be used in the methods of the invention are Fab, Fab′, F(ab′)₂, Fv, Fd, dAb, and fragments containing complementarity determining region (CDR) fragments, single-chain antibodies (scFv), chimeric antibodies, diabodies and polypeptides that contain at least a portion of an immunoglobulin that is sufficient to confer specific antigen-binding to the polypeptide (e.g., immunoadhesins).

As used herein, Fd means a fragment that consists of the V_{H} and C_{H1} domains; F_{V} means a fragment that consists of the V_{L} and V_{H} domains of a single arm of an antibody; and dAb means a fragment that consists of a V_{H} domain (Ward et al., Nature 341:544-46 (1989)). As used herein, single-chain antibody (scFv) means an antibody in which a V_{L} region and a V_{H} region are paired to form a monovalent molecules via a synthetic linker that enables them to be made as a single protein chain (Bird et al., Science 242:423-26 (1988) and Huston et al., Proc. Natl. Acad. Sci. USA 85:5879-83 (1988)). As used herein, diabody means a bispecific antibody in which V_{H} and V_{L} domains are expressed on a single polypeptide chain, but using a linker that is too short to allow for pairing between the two domains on the same chain, thereby forcing the domains to pair with complementary domains of another chain and creating two antigen-binding sites (see, e.g., Holliger et al,, Proc. Natl. Acad. Sci. USA 90:6444-48 (1993) and Poljak et al., Structure 2:1121-23 (1994)).

### Immunization

Antibodies for use in the methods of the invention can be generated by immunization of a suitable host (e.g., vertebrates, including humans, mice, rats, sheep, goats, pigs, cattle, horses, reptiles, fishes, amphibians, and in eggs of birds, reptiles and fish). Such antibodies may be polyclonal or monoclonal. For a review of methods for making antibodies see, e.g., Harlow and Lane (1988), Antibodies, A Laboratory Manual; Yelton et al., Ann. Rev. of Biochem., 50:657-80 (1981); and Ausubel et al. (1989), Current Protocols in Molecular Biology (New York: John Wiley & Sons). Determination of immunoreactivity with an immunogenic Nogo receptor polypeptide may be made by any of several methods well known in the art, including, e.g., immunoblot assay and ELISA. Monoclonal antibodies for use in the methods of the invention can be made by standard procedures as described, e.g., in Harlow and Lane (1988), supra.

For example, a host may be immunized with an immunogenic Nogo receptor-1 polypeptide either with or without an adjuvant. Suitable polypeptides are described in, for example, International Patent Applications PCT/US01/31488, PCT/US02/32007 and PCT/US03/25004. The host also may be immunized with Nogo receptor-1 associated with the cell membrane of an intact or disrupted cell and antibodies identified by binding to a Nogo receptor-1 polypeptide. Other suitable techniques for producing an antibody involve in vitro exposure of lymphocytes to the Nogo receptor-1 or to an immunogenic polypeptide of the invention, or alternatively, selection of libraries of antibodies in phage or similar vectors. See Huse et al., Science 246:1275-81 (1989).

Anti-Nogo receptor-1 antibodies used in the methods of this invention also can be isolated by screening a recombinant combinatorial antibody library. Methodologies for preparing and screening such libraries are known in the art. There are commercially available methods and materials for generating phage display libraries (e.g., the Pharmacia Recombinant Phage Antibody System, catalog no. 27-9400-01; the Stratagene SurfZAP™ phage display kit, catalog no. 240612; and others from MorphoSys). Following screening and isolation of an anti-Nogo receptor-1 antibody from a recombinant immunoglobulin display library, the nucleic acid encoding the selected antibody can be recovered from the display package (e.g., from the phage genome) and subcloned into other expression vectors by standard recombinant DNA techniques. To express an antibody isolated by screening a combinatorial library, DNA encoding the antibody heavy chain and light chain or the variable regions thereof is cloned into a recombinant expression vector and introduced into a host cell.

### Uses for Nogo Receptor Antagonists

This invention relates to methods of promoting regeneration or survival of dopaminergic neurons in a mammal displaying signs or symptoms of dopaminergic neuronal degeneration. In some embodiments of this invention, the dopaminergic neuronal degeneration is associated with a disease, disorder or condition including, but not limited to, Parkinson's disease.

In a preferred embodiment, the disease, disorder or condition is Parkinson's disease.

### Nogo Receptor Antagonist Pharmaceutical Compositions

The Nogo receptor antagonists used in the methods of the invention may be formulated into pharmaceutical compositions for administration to mammals, including humans. The pharmaceutical compositions used in the methods of this invention comprise pharmaceutically acceptable carriers.

Pharmaceutically acceptable carriers useful in these pharmaceutical compositions include, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions used in the methods of the present invention may be administered by any suitable method, e.g., parenterally, intraventricularly, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In methods of the invention, the Nogo receptor antagonist must cross the blood-brain barrier. This crossing can result from the physico-chemical properties inherent in the Nogo receptor antagonist molecule itself, from other components in a pharmaceutical formulation, or from the use of a mechanical device such as a needle, cannula or surgical instruments to breach the blood-brain barrier. Where the Nogo receptor antagonist is a soluble Nogo receptor, anti-Nogo receptor antibody, or other molecule that does not inherently cross the blood-brain barrier, a suitable route of administration is intracranial, e.g., directly into the substantia nigra or the striatum. Where the Nogo receptor antagonist is a molecule that inherently crosses the blood-brain barrier, the route of administration may be by one or more of the various routes described below.

Sterile injectable forms of the compositions used in the methods of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Parenteral formulations may be a single bolus dose, an infusion or a loading bolus dose followed with a maintenance dose. These compositions may be administered once a day or on an “as needed” basis.

Certain pharmaceutical compositions used in the methods of this invention may be orally administered in any orally acceptable dosage form including, e.g., capsules, tablets, aqueous suspensions or solutions. Certain pharmaceutical compositions also may be administered by nasal aerosol or inhalation. Such compositions may be prepared as solutions in saline, employing benzyl alcohol or other suitable preservatives, absorption promoters to enhance bioavailability, fluorocarbons, and/or other conventional solubilizing or dispersing agents.

The amount of Nogo receptor antagonists that may be combined with the carrier materials to produce a single dosage form will vary depending upon the host treated and the particular mode of administration. The composition may be administered as a single dose, multiple doses or over an established period of time in an infusion. Dosage regimens also may be adjusted to provide the optimum desired response (e.g., a therapeutic or prophylactic response).

The methods of the invention use a “therapeutically effective amount” or a “prophylactically effective amount” of a Nogo receptor antagonist. A therapeutically or prophylactically effective amount of the Nogo receptor antagonist used in the methods of the invention may vary according to factors such as the disease state, age, sex, and weight of the individual. A therapeutically or prophylactically effective amount is also one in which any toxic or detrimental effects are outweighed by the therapeutically beneficial effects.

A specific dosage and treatment regimen for any particular patient will depend upon a variety of factors, including the particular Nogo receptor antagonist, the patient's age, body weight, general health, sex, and diet, and the time of administration, rate of excretion, drug combination, and the severity of the particular disease being treated. Judgment of such factors by medical caregivers is within ordinary skill in the art. The amount of antagonist will also depend on the individual patient to be treated, the route of administration, the type of formulation, the characteristics of the compound used, the severity of the disease, and the desired effect. The amounts of antagonists can be determined by pharmacological and pharmacokinetic principles well-known in the art.

In the methods of the invention, the Nogo receptor antagonists are generally administered intracerebroventricularly, intrathecally or directly to the central nervous system (CNS), e.g. into the midbrain, substantia nigra or striatum. Compositions for administration according to the methods of the invention can be formulated so that a dosage of 0.001-10 mg/kg body weight per day of the Nogo receptor antagonist is administered. In some embodiments of the invention, the dosage is 0.01-1.0 mg/kg body weight per day. In some embodiments, the dosage is 0.05-0.5 mg/kg body weight per day.

Supplementary active compounds also can be incorporated into the compositions used in the methods of the invention. For example, a Nogo receptor antibody or an antigen-binding fragment thereof, or a soluble Nogo receptor polypeptide or a fusion protein may be coformulated with and/or coadministered with one or more additional therapeutic agents.

The invention encompasses any suitable delivery method for a Nogo receptor antagonist to a selected target tissue, including bolus injection of an aqueous solution of a Nogo receptor antagonist or implantation of a controlled-release system. Use of a controlled-release implant reduces the need for repeat injections.

The Nogo receptor antagonists used in the methods of the invention may be directly infused into the brain. Various implants for direct brain infusion of compounds are known and are effective in the delivery of therapeutic compounds to human patients suffering from neurological disorders. These include chronic infusion into the brain using a pump, stereotactically implanted, temporary interstitial catheters, permanent intracranial catheter implants, and sugically implanted biodegradable implants. See, e.g., Gill et al., supra; Scharfen et al., “High Activity Iodine-125 Interstitial Implant For Gliomas,” Int. J. Radiation Oncology Biol. Phys. 24(4):583-91 (1992); Gaspar et al., “Permanent ¹²⁵I Implants for Recurrent Malignant Gliomas,” Int. J. Radiation Oncology Biol. Phys. 43(5):977-82 (1999); chapter 66, pages 577-580, Bellezza et al., “Stereotactic Interstitial Brachytherapy,” in Gildenberg et al., Textbook of Stereotactic and Functional Neurosurgery, McGraw-Hill (1998); and Brem et al., “The Safety of Interstitial Chemotherapy with BCNU-Loaded Polymer Followed by Radiation Therapy in the Treatment of Newly Diagnosed Malignant Gliomas: Phase I Trial,” J. Neuro-Oncology 26:111-23 (1995).

The compositions may also comprise a Nogo receptor antagonist dispersed in a biocompatible carrier material that functions as a suitable delivery or support system for the compounds. Suitable examples of sustained release carriers include semipermeable polymer matrices in the form of shaped articles such as suppositories or capsules. Implantable or microcapsular sustained release matrices include polylactides (U.S. Pat. No. 3,773,319; EP 58,481), copolymers of L-glutamic acid and gamma-ethyl-L-glutamate (Sidman et al., Biopolymers 22:547-56 (1985)); poly(2-hydroxyethyl-methacrylate), ethylene vinyl acetate (Langer et al., J. Biomed. Mater. Res. 15:167-277 (1981); Langer, Chem. Tech. 12:98-105 (1982)) or poly-D-(−)-3hydroxybutyric acid (EP 133,988).

In some embodiments of the invention, a Nogo receptor antagonist is administered to a patient by direct infusion into an appropriate region of the brain. See, e.g., Gill et al., “Direct brain infusion of glial cell line-derived neurotrophic factor in Parkinson disease,” Nature Med. 9: 589-95 (2003). Alternative techniques are available and may be applied to administer a Nogo receptor antagonist according to the invention. For example, stereotactic placement of a catheter or implant with a Nogo receptor antagonist using the Riechert-Mundinger unit and the ZD (Zamorano-Dujovny) multipurpose localizing unit can be utilized. A contrast-enhanced computerized tomography (CT) scan, injecting 120 ml of omnipaque, 350 mg iodine/ml, with 2 mm slice thickness can allow three-dimensional multiplanar treatment planning (STP, Fischer, Freiburg, Germany). This equipment permits planning on the basis of magnetic resonance imaging studies, merging the CT and MRI target information for clear target confirmation.

The Leksell stereotactic system (Downs Surgical, Inc., Decatur, Ga.) modified for use with a GE CT scanner (General Electric Company, Milwaukee, Wis.) as well as the Brown-Roberts-Wells (BRW) stereotactic system (Radionics, Burlington, Mass.) can be used for this purpose. Thus, on the morning of the implant, the annular base ring of the BRW stereotactic frame can be attached to the patient's skull. Serial CT sections can be obtained at 3 mm intervals though the (target tissue) region with a graphite rod localizer frame clamped to the base plate. A computerized treatment planning program can be run on a VAX 11/780 computer (Digital Equipment Corporation, Maynard, Mass.) using CT coordinates of the graphite rod images to map between CT space and BRW space.

### EXAMPLES

### Example 1

### Soluble Nogo Receptor (310)-Fc Reduced Rotational Behavior and Increased Striatal Dopamine Levels after 6-Hydroxydopamine Lesioning in the Rat

Male Sprague-Dawley rats (150-200 g, Charles River) were anaesthetized using isoflurane and placed in a stereotaxic frame. The surgical site was wiped with betadine and alcohol and a 1-inch midline saggittal incision made to expose bregma. A Small burr hole was made in the skull above the injection site and 20 μg 6-hydroxydopamine HCl (6-OHDA) in 2 μl (saline/0.2% ascorbate) stereotaxically infused into the left striatum at co-ordinates AP +0.7, Lateral 2.8 mm lateral to the midline, DV −5.5 mm ventral to the surface of the skull. The 6-OHDA was infused over 4 min at a rate of 0.5 μl/min using a syringe pump attached with polyethylene tubing to a 29 gauge stainless steel cannula. After infusion of the 6-OHDA, the cannula was left in place for an additional 2 min then withdrawn slowly. An alzet brain infusion cannula, 5 mm in length, was then implanted through the same burr hole and fixed to the skull using superglue. The cannula was connected to a primed Alzet osmotic pump (model 2004) containing PBS or 50 mM sNgR(310)Fc (a fusion protein comprising amino acids 26-310 of rat Nogo receptor-1 and a rat Fc fragment; see International Patent Application PCT/US03/25004) that continuously released at a rate of 0.25 μl/h for 28 days. The osmotic pump was implanted into the subcutaneous space at the scruff of the neck. The incision site was closed using autoclips and rats placed in a humidified incubator until recovery from anesthesia.

Seven, 14, 21 and 28 days after 6-OHDA infusion rats were treated with amphetamine (1 mg/kg ip) and rotational behavior measured over a 2 hour period. “Rotational behavior” is the behavior exhibited when an animal with unilateral damage to the nigrostriatal dopamine pathway is administered a dopamine agonist such as apormorphine or a dopamine releasing agent such as amphetamine. The animal repeatedly turns in circles away from the side of the brain experiencing greater striatal dopamine receptor stimulation. The magnitude of the rotational response, i.e., the number of rotations performed, is directly proportional to the degree of damage to the nigrostriatal dopamine pathway. See, e.g., Fuxe et al., “Antiparkinsonian drugs and dopaminergic neostriatial mechanisms: studies in rats with unilateral 6-hydroxydopamine-induced degeneration of the nigro-neostriatal DA Pathway and quantitative recording of rotational behaviour,” Pharmacol. Ther. [B] 2:41-47 (1976). At least 24 h after the last rotation, test rats were sacrificed by CO₂ asphyxiation. Brains were rapidly removed and cut in the coronal plane at the posterior border of the optic chiasm. Striata were dissected bilaterally from the anterior portion of the brain and frozen on dry ice for catecholamine measurement by HPLC/EC. The posterior portion of the brain was immersion fixed in 4% PFA for 48 h and transferred to 30% sucrose for cryoprotection until cryosectioning for substantia nigra tyrosine hydroxylase immunohistochemistry.

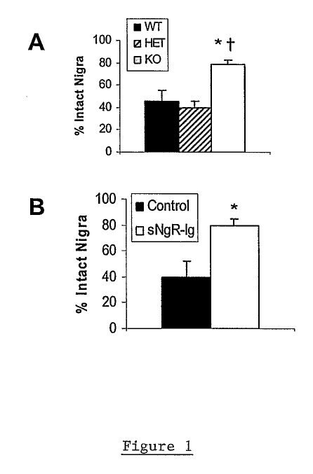

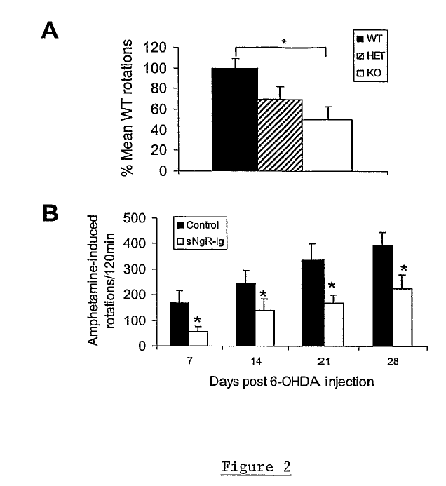

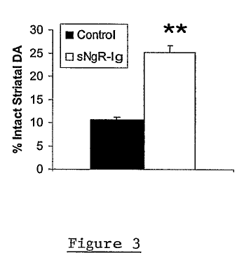

Treatment with sNgR(310)Fc significantly increased dopaminergic neuronal survival in the substantia nigra (FIG. 1B) and significantly reduced rotational behavior in response to amphetamine challenge after striatal 6-OHDA lesioning (FIG. 2B). Dopamine levels were significantly increased in the lesioned striatum of sNgR(310)-Fc treated rats compared to controls (FIG. 3). Dopamine levels in the intact striatum were not significantly altered after sNgR(310)Fc treatment. These data show that treatment with the Nogo receptor antagonist sNgR(310)-Fc increased cell survival and improved recovery in dopaminergic pathways in the brain after injury.

### Example 2

### Reduced Rotational Behavior in Response to Apomorphine Challenge in NgR Null Mice after 6-OHDA Lesioning of the Striatum

Male or female Nogo receptor knockout mice, heterozygote and wild-type littermates (15-30 g) were anesthetized using ketamine and xylazine (100 and 10 mg/kg ip, respectively) and placed in a stereotaxic frame. The surgical site was wiped with betadine and alcohol and a 0.5 cm midline saggittal incision made to expose bregma. A Small burr hole was made in the skull above the injection site and 10 μg 6-hydroxydopamine HCl (6-OHDA) in 1 μl (saline/0.2% ascorbate) stereotaxically infused into the left striatum at co-ordinates AP+0.7, Lateral 2.8 mm, lateral to the midline, DV −5.5 mm ventral to the surface of the skull. The 6-OHDA was infused over 2 min at a rate of 0.5 μl/min using a syringe pump attached with polyethylene tubing to a 29 gauge stainless steel cannula. After infusion of the 6-OHDA, the cannula was left in place for an additional 2 min then withdrawn slowly. The incision was closed using wound clips and mice were placed on a warming pad until recovery from anesthesia. Twenty-eight days after 6-OHDA infusion mice were injected with apomorphine and rotational behavior recorded over a 30 min period. At least 24 h after measurement of rotational behavior mice were euthanized by CO₂ asphyxiation. Brains were rapidly removed and cut in the coronal plane at the posterior border of the optic chiasm. The posterior portion of the brain was immersion fixed in 4% PFA for 48 h and transferred to 30% sucrose for cryoprotection until cryosectioning for substantia nigra tyrosine hydroxylase immunohistochemistry.

The number of surviving dopaminergic neurons in the substantia nigra was significantly greater in Nogo receptor knockout mice compared to their heterozygote and wild-type littermate controls 4 weeks after unilateral 6OHDA injection (FIG. 1A). Rotational behavior in response to apomorphine challenge was significantly lower in NgR null mice compared to heterozygote and wildtype littermate controls (FIG. 2A). These data show increased neuronal survival and improved recovery of function in dopaminergic pathways in the brain after injury in mice lacking NgR1.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of promoting regeneration or survival of dopaminergic neurons in a mammal displaying signs or symptoms of dopaminergic neuronal degeneration, comprising administering to the mammal a therapeutically effective amount of an NgR1 antagonist.

2. The method of claim 1, wherein the NgR1 antagonist is administered directly into the central nervous system.

3. The method of claim 2, wherein the NgR1 antagonist is administered directly into the substantia nigra or the striatum.

4. The method of claim 2, wherein the NgR1 antagonist is administered by bolus injection or chronic infusion.

5. The method of claim 1, wherein the NgR1 antagonist comprises a soluble form of a mammalian NgR1.

6. The method of claim 5, wherein the soluble form of a mammalian NgR1 comprises a peptide selected from the group consisting of:
(a) amino acids 26 to 310 of human NgR1 (SEQ ID NO:3) with up to ten conservative amino acid substitutions;
(b) amino acids 26 to 344 of human NgR1 (SEQ ID NO:4) with up to ten conservative amino acid substitutions;
(c) amino acids 27 to 310 of rat NgR1 (SEQ ID NO:5) with up to ten conservative amino acid substitutions; and
(d) amino acids 27 to 344 of rat NgR1 (SEQ ID NO:6) with up to ten conservative amino acid substitutions.

7-9. (canceled)

10. The method of claim 5, wherein the soluble form of a mammalian NgR1 further comprises a fusion moiety.

11. The method of claim 10, wherein the fusion moiety is an immunoglobulin moiety.

12. The method of claim 11, wherein the immunoglobulin moiety is an Fc moiety.

13. The method of claim 1, wherein the NgR1 antagonist comprises an antibody or antigen-binding fragment thereof that binds to a mammalian NgR1.

14. The method of claim 13, wherein the antibody is selected from the group consisting of a polyclonal antibody, a monoclonal antibody, a Fab fragment, a Fab′ fragment, a F(ab′)₂ fragment, an Fv fragment, an Fd fragment, a diabody, and a single-chain antibody.

15. The method of claim 13, wherein the antibody or antigen-binding fragment thereof binds to an polypeptide bound by a monoclonal antibody produced by a hybridoma selected from the group consisting of: HB 7E11 (ATCC® accession No. PTA-4587), HB 1H2 (ATCC® accession No. PTA-4584), HB 3G5 (ATCC® accession No. PTA-4586), HB 5B10 (ATCC® accession No. PTA-4588) and HB 2F7 (ATCC® accession No. PTA-4585).

16. The method of claim 15, wherein said monoclonal antibody is produced by the HB 7E11 hybridoma.

17. The method of claim 16, wherein the polypeptide comprises an amino acid sequence selected from the group consisting of:

| | |
|---|---|
| AAAFTGLTLLEQLDLSDNAQLR; | (SEQ ID NO: 7) |
| LDLSDNAQLR; | (SEQ ID NO: 8) |
| LDLSDDAELR; | (SEQ ID NO: 9) |
| LDLASDNAQLR; | (SEQ ID NO: 10) |
| LDLASDDAELR; | (SEQ ID NO: 11) |
| LDALSDNAQLR; | (SEQ ID NO: 12) |
| LDALSDDAELR; | (SEQ ID NO: 13) |
| LDLSSDNAQLR; | (SEQ ID NO: 14) |
| LDLSSDEAELR; | (SEQ ID NO: 15) |
| DNAQLRVVDPTT; | (SEQ ID NO: 16) |
| DNAQLR; | (SEQ ID NO: 17) |
| ADLSDNAQLRVVDPTT; | (SEQ ID NO: 18) |
| LALSDNAQLRVVDPTT; | (SEQ ID NO: 19) |
| LDLSDNAALRVVDPTT; | (SEQ ID NO: 20) |
| LDLSDNAQLHVVDPTT; | (SEQ ID NO: 21) |
| and | |
| LDLSDNAQLAVVDPTT. | (SEQ ID NO: 22) |

18. The method of claim 16, wherein the polypeptide consists of an amino acid sequence selected from the group consisting of:

| | |
|---|---|
| AAAFTGLTLLEQLDLSDNAQLR; | (SEQ ID NO: 7) |
| LDLSDNAQLR; | (SEQ ID NO: 8) |
| LDLSDDAELR; | (SEQ ID NO: 9) |
| LDLASDNAQLR; | (SEQ ID NO: 10) |
| LDLASDDAELR; | (SEQ ID NO: 11) |
| LDALSDNAQLR; | (SEQ ID NO: 12) |
| LDALSDDAELR; | (SEQ ID NO: 13) |
| LDLSSDNAQLR; | (SEQ ID NO: 14) |
| LDLSSDEAELR; | (SEQ ID NO: 15) |
| DNAQLRVVDPTT; | (SEQ ID NO: 16) |
| DNAQLR; | (SEQ ID NO: 17) |
| ADLSDNAQLRVVDPTT; | (SEQ ID NO: 18) |
| LALSDNAQLRVVDPTT; | (SEQ ID NO: 19) |
| LDLSDNAALRVVDPTT; | (SEQ ID NO: 20) |
| LDLSDNAQLHVVDPTT; | (SEQ ID NO: 21) |
| and | |
| LDLSDNAQLAVVDPTT. | (SEQ ID NO: 22) |

19. The method of claim 1, wherein the therapeutically effective amount is from 0.001 mg/kg to 10 mg/kg.

20. The method of claim 19, wherein the therapeutically effective amount is from 0.01 mg/kg to 1.0 mg/kg.

21. The method of claim 20, wherein the therapeutically effective amount is from 0.05 mg/kg to 0.5 mg/kg.

22. A method of claim 1, wherein the dopaminergic neuronal degeneration is associated with a disease or disorder selected from the group consisting of Parkinson's disease, multiple system atrophy, striatonigral degeneration, olivopontocerebellar atrophy, Shy-Drager syndrome, motor neuron disease with parkinsonian features, Lewy body dementia, progressive supranuclear palsy, cortical-basal ganglionic degeneration, frontotemporal dementia, Alzheimer's disease with parkinsonism, Wilson disease, Hallervorden-Spatz disease, Chediak-Hagashi disease, SCA-3 spinocerebellar ataxia, X-linked dystonia-parkinsonism (DYT3), Huntington's disease (Westphal variant), prion disease, vascular parkinsonism, cerebral palsy, repeated head trauma, postencephalitic parkinsonism and neurosyphilis.

23. A method of treating Parkinson's disease, comprising administering to the mammal a therapeutically effective amount of an NgR1 antagonist.
